(19) United States
(12) Patent Application Publication
(10) Pub. No.: US 2011/0022131 A1
(43) Pub. Date: Jan. 27, 2011

(54) Method for Restoring an Ejaculatory Failure

(75) Inventor: Francois Giuliano, Saint-Cloud (FR)

Correspondence Address: FULBRIGHT & JAWORSKI L.L.P., 600 CONGRESS AVE., SUITE 2400, AUSTIN, TX, 78701, US

(73) Assignee: PELVIPHARM, Orsay (FR)

(21) Appl. No.: 12/936,040

(22) PCT Filed: Apr. 1, 2009
(86) PCT No.: PCT/EP09/53909

§ 371 Date: Oct. 1, 2010

Related U.S. Application Data
(60) Provisional application No. 61/041,273, filed on Apr. 1, 2008.

(51) Int. Cl. A61N 5/06; C12N 7/00

(52) U.S. Cl. 607/88; 435/235.1

(57) ABSTRACT

The present invention relates to a method for eliciting ejaculation in a male individual, comprising delivering one or more stimulation pulses to lumbar spinothalamic (LSt) cells via a light stimulus, wherein said LSt cells express a light-activated cation channel protein.

### FIELD OF THE INVENTION

The present invention relates to methods for eliciting ejaculation in a male individual possibly suffering from an ejaculation failure.

### BACKGROUND OF THE INVENTION

Sexual performance in humans involves many functions: in males, these functions mainly are erection, ejaculation and orgasm. Ejaculation is not to be confounded with orgasm from a physiological perspective: in particular, in spinal cord injured patients ejaculation can be achieved without orgasm. A wide variety of medical and psychological problems may also interfere with one or more of these functions.

Methods to treat these sexual dysfunctions are known in the art. For example, U.S. Pat. No. 6,169,924 describes stimulation of the spinal cord to achieve orgasm, whereas to achieve erection, US2005/0222628 patent application describes stimulation of the pelvic nerve and US2005/0096709 patent application describes electrical stimulation of the prostate gland.

Ejaculation comprises two distinct and successive phases: emission and expulsion. Emission involves transport of spermatozoa from the epidydimis along the vas deferens and their mixing with secretions from prostate and seminal vesicles (semen) before terminating as sperm in the prostatic urethra. Expulsion is the forceful expulsion of sperm from the urethra out of the urethral meatus and depends on the coordinated and rhythmic contraction of the striated perineal muscles, in particular the bulbospongiosus muscle. Ejaculation is thus a complex mechanism, and the prior art failed in proposing solutions for eliciting simultaneously the whole process: US2005/0222628 patent application describes stimulation of the pelvic plexus nerves to achieve emission but fails to address the expulsion issue; US2005/0096709 patent application describes electrical stimulation of the prostate gland to achieve ejaculation, but electrical stimulation of the prostate gland only causes emission and not expulsion. Therefore, known treatments for ejaculation failure allow only the first phase of ejaculation i.e. emission but do not lead to a complete ejaculation with expulsion of the sperm.

Recently, new data have been published providing a better comprehension of the mechanism of ejaculation. Ejaculation can occur in response to genital stimulation in humans and rats after complete lesion of the spinal cord above thoracic segment 10 (T10), evidencing that the spinal cord is still able to command and organize the peripheral events leading to ejaculation. In rats, lumbar spinothalamic (LSt) neurons in lamina VII and X of the lumbar spinal segment L3-L4 have been postulated to form a spinal generator for ejaculation (SGE) to coordinate the sympathetic, parasympathetic and somatic efferent activities (Truitt and Coolen, 2002, Science 297:1566). During copulation in rats, the expression of a marker for neuronal activity, c-Fos, increases in L3-L4 LSt neurons after ejaculation and not after mounts and intromissions (Truitt and Coolen, 2003, J. Neurosci. 23:325).

Coolen et al (US2004/0152631) mention a method comprising the administration to an individual of a drug such as neurotransmitters, for example gamma-amino-butyric acid, or neuropeptides for example serotonin, galanin, somatostatin, which may interact with LSt cells. They suggest that this method would allow manipulation of the sensation of ejaculation; however, they do not prove that it could lead to the restoration of an ejaculation failure.

Nevertheless, LSt neurons still provide an interesting target for eliciting ejaculation, and, taking into account that chemical drugs may induce undesirable side effects, the Applicant focussed on alternatives means to medication, for eliciting ejaculation or restoring an ejaculation failure, such as for example anejaculation, which is a common ejaculatory dysfunction in spinal-cord-injured men.

### SUMMARY OF THE INVENTION

The invention relates to a method for eliciting ejaculation in a male individual, comprising delivering one or more stimulation pulses to LSt cells via a light stimulus, in an effective amount to activate LSt cells for achieving expulsion of sperm, wherein said LSt cells express a light-activated cation channel protein.

According to an embodiment, said light-activated cation channel protein is selected among ChR2, Chop2, ChR2-310, Chop2-310 and fragments or derivatives thereof.

According to another embodiment, said light stimulus is provided by a xenon lamp or a laser. In an embodiment of the invention, the level of light intensity is from 0.1 mW/mm² to 500 mW/mm². In another embodiment, the wavelength of the light stimulus is from 400 nm to 600 nm. In another embodiment, said light stimulus is provided in a series of light pulses having a period from 0.1 ms to 100 ms.

In an embodiment of the invention, said light stimulus is provided by a wearable optical device. In a preferred embodiment, said wearable optical device is a light-emitting diode. According to an embodiment of the method of the invention, the individual suffers from an ejaculation failure.

Another object of the invention is an adeno-associated virus of serotype 2/8 (AAV2/8) comprising a light-activated cation channel protein.

In one embodiment, said light-activated cation channel protein is ChR2, Chop2, Chr2-310 or Chop2-310.

In another embodiment, said light-activated cation channel protein is encoded by SEQ ID NO: 1 or SEQ ID NO: 2.

### DETAILED DESCRIPTION OF THE INVENTION

The present invention thus relates to a method for eliciting ejaculation in a male individual or for restoring an ejaculation failure in a male individual suffering there from, comprising delivering one or more stimulation pulses to lumbar spinothalamic (LSt) cells via a suitable device, in an effective amount to activate LSt cells for achieving expulsion of sperm. In the meaning of the present invention, a male individual refers to a male human being or a male animal, preferably a mammal. Preferably, a male individual means a man or a boy over 16. In a first embodiment, said male individual is suffering from an ejaculation failure. In a second embodiment, said male individual is not suffering from an ejaculation failure.

Restoring an ejaculation failure may be considered as a medical need and in the embodiment of the invention where the male individual of the invention necessitates a restoration of ejaculation functions, the device used in this invention may be considered as a medical device. Example of male individual necessitating a restoration of ejaculation functions are spinal-cord-injured men suffering from anejaculation.

However, according to another embodiment, this invention may also be useful for eliciting ejaculation in males which are not suffering from a medically-recognized deprivation/impairment of their ejaculation, and in this embodiment, the device of the invention shall be considered as a personal healthcare device.

In the meaning of this invention “ejaculation” comprises two distinct and successive phases: emission and expulsion of sperm.

The inventors showed that eliciting ejaculation in a male individual or restoring an ejaculation failure in a male individual suffering there from can be achieved by delivering one or more electric pulses delivered by electric means placed in the area of LSt cells (see results).

One object of the present invention is to provide a method for eliciting ejaculation in a male individual, comprising delivering one or more stimulation pulses to LSt cells via a light stimulus, in an effective amount to activate LSt cells for achieving expulsion of sperm, said LSt cells expressing a light-activated cation channel protein.

Without willing to be bound with a theory, the Applicant submits that light-illumination of LSt neurons expressing light-activated cation channel proteins will shift the transmembrane electrical potential across the LSt cells' outer cell membrane to a more positive value, thereby activating the LSt cells.

According to the invention, said light-activated cation protein comprises channelrhodopsin-2 (ChR2) or Channelopsin-2 (Chop2) (encoded by the gene referred in Genbank accession No. AF461397), a synthetic form of ChR2 gene optimized for expression in mammals (encoded by the gene referred in Genbank accession No. EF474017.1), and fragments thereof. In another embodiment, it also encompasses channelrhodopsin-1. Said light-activated cation protein are described in WO2007/024391 which is incorporated herein by reference.

ChR2 is a rhodopsin derived from the unicellular green alga Chlamydomonas reinhardtii. The term “rhodopsin” as used herein is a protein that comprises at least two buildings blocks, an opsin protein and a covalently bound cofactor, usually retinal. The term “retinal”, as used herein, comprises all-trans retinal, 11 cis-retinal, and others isomers of retinal. The term “ChR2” or “Chop2” as used herein refers to the full proteins or fragments thereof.

In a preferred embodiment of the invention, a fragment comprising the amino terminal 310 amino acids of ChR2 or Chop2 is used.

“Protein” as used herein includes proteins, polypeptides, and peptides. Also included within the light-activated cation channel protein of the present invention are amino acid variants of the naturally occurring sequences, as determined herein. Preferably, the variants are greater than about 75% homologous to the protein sequence of Chop2, ChR2, Chop2-310 or ChR2-310, more preferably greater than about 80%, even more preferably greater than about 85% and most preferably greater than 90%. In some embodiments the homology will be as high as about 93 to about 95 or about 98%. Homology in this context means sequence similarity or identity, with identity being preferred. This homology will be determined using standard techniques known in the art.

In one embodiment of the invention, the light-activated cation channel proteins used in the invention are derivative or variant protein sequences, as compared to Chop2 ChR2, Chop2-310 or ChR2-310. That is, the derivative proteins of the invention will contain at least one amino acid substitution, deletion or insertion, with amino acid substitutions being particularly preferred. The amino acid substitution, insertion or deletion may occur at any residue within the protein. These variants or derivatives are ordinarily prepared by site specific mutagenesis of nucleotides in the DNA encoding the light-activated cation channel proteins, using cassette or PCR mutagenesis or other techniques well known in the art, to produce DNA encoding the variant, and thereafter expressing the DNA in recombinant cell culture. The variants or derivatives typically exhibit the same qualitative biological activity as Chop2 ChR2, Chop2-310 or ChR2-310, or an optimised qualitative biological activity compared to Chop2 ChR2, Chop2-310 or ChR2-310. For example, the protein can be modified such that it can be driven by different wavelength of light than the wavelength of around 460 nm of the wild type ChR2 protein. The protein can be modified, for example, such that it can be driven at a longer wavelength such as about 480 nm, 490 nm, 500 nm, 510 nm, 520 nm, 530 nm, 540 nm, 550 nm, 560 nm, 570 nm, 580 nm, or 590 nm. According to another embodiment, the light-activated cation channel protein may be comprised in a fusion protein, said fusion protein being used to target the light-activated cation channel protein to LSt cells or specific regions within LSt cells. For example, a PDZ domain may be used to target dendrites and an AIS domain may be used to target axons.

According to the invention, the light-activated cation channel protein disclosed here above is contained in a vector, in order to express said protein in LSt neurons. As used herein the term “vector” refers to a nucleic acid molecule capable of transporting between different genetic environments another nucleic acid to which it has been operatively linked. Examples of vectors are viruses such as lentiviruses, retroviruses, adenoviruses and phages.

According to a novel embodiment of the invention, the vector comprising the light-activated cation channel protein disclosed here above is the adeno-associated virus of serotype 2/8: AAV 2/8.

An object of the invention is an adeno-associated virus of serotype 2/8 comprising a light-activated cation channel protein.

In one embodiment, said light-activated cation channel protein is ChR2, Chop2, ChR2-310 or Chop2-310.

According to this embodiment, said light-activated cation protein may be channelrhodopsin-2 (ChR2) or Channelopsin-2 (Chop2) (encoded by the gene referred in Genbank accession No. AF461397), a synthetic form of ChR2 gene optimized for expression in mammals (encoded by the gene referred in Genbank accession No. EF474017.1), and fragments thereof, for example amino acids 2 to 310 of ChR2 or Chop2. In one embodiment, said light-activated cation protein is encoded by SEQ ID No: 1 or SEQ ID NO: 2.

In one embodiment, said adeno-associated virus of serotype 2/8 comprising a light-activated cation channel protein is in a pharmaceutically acceptable carrier.

In a preferred embodiment of the invention, the nucleic acid coding the light-activated cation channel protein or fragment thereof is operatively linked to a promoter and contained in a lentivirus or a retrovirus. Examples of promoters include, but are not limited to, neuron specific promoters such as enolase promoter, promoters for cholecystokinin, somatostatin, parvalbumin, GABA□6, L7, calbindin, EF1-□, promoters for kinases such as PKC, PKA, and CaMKII; promoters for other ligand receptors such as NMDAR1, NMDAR2B, GluR2; promoters for ion channels including calcium channels, potassium channels, chloride channels, and sodium channels; and promoters for other markers that label classical mature and dividing cell types, such as calretinin, nestin, and beta3-tubulin.

According to the invention, LSt cells are targeted by the vector as described here above to express light-activated cation channel proteins.

LSt cells can be found in the area of lumbar spinothalamic L1 to L4 segments, preferably in the area of L2 to L4 segments. More precisely, LSt cells can be found in lamina VII and X of lumbar spinothalamic L1 to L4 segments.

In one embodiment of the invention, LSt cells of the subject are targeted in vivo with a vector as described here above allowing the expression of light-activated cation channel proteins in LSt cells. According to this embodiment, a therapeutically effective amount of said vector, preferably of AAV2/8, is injected to a subject in need thereof. In one embodiment, said injection is carried out by an intraspinal route.

Those skilled in the art will be familiar with the preparation of functional AAV-based gene therapy vectors. Numerous references to various methods of AAV production, purification and preparation for administration to human subjects can be found in the extensive body of published literature (see, e.g., Viral Vectors for Gene Therapy: Methods and Protocols, ed. Machida, Humana Press, 2003)

In another embodiment, LSt cells are targeted ex vivo with a vector as described here above allowing the expression of light-activated cation channel proteins in said cells and then re-implanted in the subject they come from.

According to the invention, said adeno-associated virus of serotype 2/8 (AAV2/8) as described here above is for eliciting ejaculation in a male individual.

According to the invention, said adeno-associated virus of serotype 2/8 (AAV2/8) as described here above is for use in eliciting ejaculation in a male individual.

According to the invention, said adeno-associated virus of serotype 2/8 (AAV2/8) as described here above is for treating an ejaculation failure.

According to the invention, said adeno-associated virus of serotype 2/8 (AAV2/8) as described here above is for use in treating an ejaculation failure.

According to these embodiment, said adeno-associated virus of serotype 2/8 (AAV2/8) as described here above is to be administrated to a subject, in order to target LSt cells.

According to the invention, said one or more stimulation pulses are delivered to LSt cells via a light stimulus.

Preferably, said stimulation pulses are delivered to the area of lumbar spinothalamic L1 to L4 segments, more preferably to the area of L2 to L4 segments.

In a preferred embodiment of the invention, said stimulation pulses are delivered to lamina VII and X of lumbar spinothalamic L1 to L4 segments where LSt cells are located.

According to an embodiment of the invention, the light stimulus used to deliver stimulation pulses to LSt cells is provided by a xenon lamp, a light-emitting diode (LED) or a laser. The light intensity used is chosen not to damage the cells. Thus, a medium intensity light is used.

In a preferred embodiment, the level of light is from 0.1 mW/mm² to 500 mW/mm², preferably from 1 mW/mm² to 100 mW/mm² and most preferably from 5 mW/mm² to 50 mW/mm².

In a preferred embodiment, the wavelength of the illuminating light is from 400 nm to 600 nm or is suitable to activate the light-activated cation channel protein.

Preferably, the wavelength of the illuminating light is from 450 nm to 550 nm and more preferably from 450 nm to 490 nm.

In a preferred embodiment, said stimulation pulses are delivered by a series of light pulses in which light period are from 0.1 ms to 100 ms, preferably from 1 ms to 50 ms, most preferably from 5 ms to 20 ms. Such rapid light pulses may be followed by a period of darkness. The period of darkness can be greater than 1 ms, preferably greater than 10 ms, most preferably greater than 20 ms or can be longer if desired.

In a preferred embodiment, the light used to deliver stimulation pulses is blue light.

In one embodiment of the invention, the light used to deliver stimulation pulses to LSt cells can come from a wearable optical device. Such optical wearable optical device may be for example implantable under the skin at the level of lumbar spinothalamic L1 to L4 segments.

In another embodiment of the invention, the light used to deliver stimulation pulses to LSt cells can come from a fixed optical station.

In an embodiment of the invention, the optical device used to deliver stimulation pulses to LSt cells is a light-emitting diode (LED). The LED can be of millimetre to nanometer scale size. An example of such LED is SML0805-B1K-TR LEDtronics (which emits 460 nm wavelength light).

The LED can be battery-powered or remotely powered. A remotely-powered LED could be made by combining a LED in a closed-loop series circuit with an inductor. This would allow radio frequency energy or rapidly changing magnetic fields to temporarily power-up the inductor, and thus the connected LED, allowing local delivery of light.

It is understood that the examples described here after are presented for illustrating the invention.

### DETAILED DESCRIPTION OF THE DRAWINGS

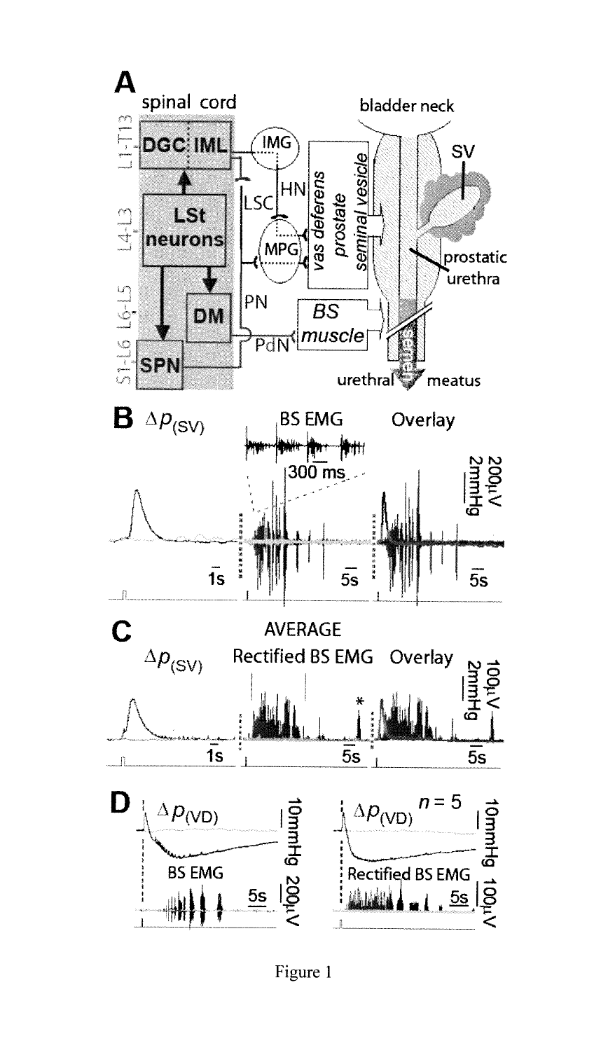

FIG. 1: Ejaculation-related events elicited by electrical microstimulation of LSt neurons.

(A) Schematic representation of connections of LSt neurons with pelvi-perineal anatomical structures involved in rat ejaculation. DGC: dorsal gray commisure; IML: intermediolateral column; DM: dorso-medial part of Ones nucleus; SPN: sacral parasympathetic nucleus; HN: hypogastric nerve; LSC: lumbosacral paravertebral sympathetic chain; PN: pelvic nerve; PdN: pudendal nerve; IMG: intermesenteric ganglion; MPG: major pelvic ganglion; BS: bulbospongiosus, SV: seminal vesicle. Spinal level for each nucleus is indicated in gray.

(B) Simultaneous recording of Δp_{(SV)} (dark gray) and BS EMG (black) elicited by LSt neuron microstimulation in an anesthetized rat. Stimulation protocol: 300 ms of 0.5 ms biphasic current pulses repeated at 200 Hz (60 pulses). Stimulation amplitude: ≧3 times the Δp_{(SV)} response threshold. Light gray traces: background activity. The overlay (right) displays the sequential activation of SV and BS muscle after LSt neuron microstimulation. Inset, middle panel: evoked BS EMG on an expanded timescale shows 4 bursts within the BS EMG response, demonstrating the regular rhythmic bursting pattern.

(C) Averaged response for recordings from 7 animals (experimental protocol and color codes as in B). BS EMG is shown rectified and 200 Hz low-pass filtered. Vertical lines: onset of BS EMG activity and time when 95% of BS EMG activity has occurred. Asterisk: late burst of BS EMG activity after LSt neuron microstimulation.

(D) Simultaneous recording of Δp_{(VD)} (dark gray) and BS EMG (black) elicited by LSt neuron microstimulation [stimulation protocol as in B]. Left panel: example experiment. Right panel: averaged response for recordings from 5 animals. Light gray traces: background activity.

### EXAMPLES

### 1-Electrical Stimulation

### Materials and Methods

### Animals and Surgery

All procedures were in accordance with the European Communities Council Directives 86/609/EEC on the use of laboratory animals. Male adult sexually naïve Wistar rats (Janvier, Le Genest-St-Isle, France) of 275-325 grams, housed for at least 4 days in our animal facility before experimentation, were anesthetized with 1.2 mg/kg intraperitoneal urethane while body temperature was maintained at 37° C. with a homeothermic blanket. Paw withdrawal and eye blink reflexes were largely suppressed. Custom-made bipolar steel wire electrodes (AS631, CoonerWire, Calif., USA) were implanted into the exposed dorsal part of the right bulbospongiosus (BS) muscle. After suprapubic midline abdominal incision, a 1.1 mm diameter mineral oil-filled catheter was inserted into the lumen of the right seminal vesicle (SV) via its apex or the right vas deferens (VD) was cut and a 0.61 mm diameter catheter filled with isotonic salt solution inserted into the prostatic portion of the VD lumen. Then, the spine was exposed dorsally and fixed with a stereotaxic frame. Laminectomy was performed between vertebrae L1-T13 to expose L4 spinal level and the dura was carefully removed. To improve intraspinal access, we incubated the spinal cord for 20 minutes with 3 units/μl collagenase type VII from Clostridium histolyticum (Sigma-Aldrich Chimie, St. Quentin Fallavier, France).

### Spinal Microstimulation

Monopolar spinal microstimulation was performed with a ‘Formvar’-coated nichrome wire of 50 μm diameter (AM-Systems Inc. WA, USA). Typically, the electrode was positioned on the dorsal surface at L4 spinal level, adjacent to the right of the dorsal spinal artery and lowered vertically with a hydraulic microdrive (Trent-Wells, Coulterville, Calif., USA) to ˜1600 μm depth in correspondence with the stereotaxic coordinates for laminae VII and X (S2), taking as electrode depth the read-out of the microdrive. A reference electrode was placed in the vicinity of the tail. Electrical stimuli were delivered using a pulse generator (model-2100, AM-Systems Inc. WA, USA). Biphasic rectangular current pulses of 0.5 ms duration applied in short trains of 60 to 100 pulses at 200 Hz for a total duration of 300 to 500 ms were applied. This stimulus was optimal without causing temporal overlap between stimulus and the SV/VD/BS muscle responses, according to preliminary experiments. The stimulation amplitude was set to ≧3 times the threshold for eliciting an SV, VD and/or BS response (15-100 μA). For each stimulation, the ejaculate was collected on a coverslip and directly put under the microscope (Olympus CH-2, Olympus SAS, France; magnification 40×) in order to detect and observe spermatozoa. Sometimes, but not always, we observed BS EMG activity during the time of microstimulation, often associated with hind leg movements.

### Verification of the Lateral Position of the Spinal Microstimulation Electrode

At the end of the experiment, spinal cord tissue was lesioned with 2-3 repeats of 1-2 mA current injections through the electrode used in the LSt stimulation protocol. The animal was then perfused transcardiacally for 15 minutes with ˜600 ml 4% paraformaldehyde, the spinal cord removed and sliced into 30 μm thick slices with a cryostat. The shortest distance between the centre of the lesion and the spinal cord midline was taken as the electrode lateral position.

### BS Muscle EMG and Intraluminal SV/VD Pressure Change Recording

EMG from the proximal part of the BS muscle (BS EMG), was recorded differentially, amplified and filtered (model-1700, AM-Systems Inc., USA; amplification 1000×, bandpass filter settings 0.1-1 kHz). To quantify SV contraction, luminal SV pressure change (Δp_{(SV)}) was measured at the tip of the oil-filled tube (total length ˜200 mm) with a pressure sensor (26PCAFG6G, Honeywell Inc., USA) connected to a bridge amplifier (TRN005, Kent Scientific Corp., UK; amplification 1000× or 2000×, 100 Hz lowpass filter). In preliminary experiments, we confirmed that Δp_{(SV)} recorded with this technique closely related to in situ values measured simultaneously with a miniature pressure probe (SambaSensors SAB, Sweden), with only ˜5% error in absolute values. To quantify VD contraction, luminal VD pressure change (Δp_{(VD)}) was measured at the tip of the tube (total length ˜300 mm, filled with isotonic salt solution) with a pressure sensor. Basal VD luminal pressure was increased to 37±4 mmHg (n=5) through continuous perfusion of the tube with isotonic salt solution at a rate of 2.25 μl*min⁻¹. This procedure aimed to prevent obstruction of the tube tip, but also explained the decrease in VD pressure after VD contraction as seen in FIG. 1 D, reflecting refilling of the VD with isotonic salt solution. Data was stored at 5 kHz sampling rate on a PC for later analysis.

### Data Analysis

BS EMG, Δp_{(SV)} and Δp_{(VD)} recordings were analyzed using custom written routines in Elphy software (G. Sadoc, CNRS, Gif-sur-Yvette, France). Mean baseline values over 1 s before microstimulation were subtracted from each recording trace before analysis. For BS EMG quantification, EMG signals were rectified, 200 Hz lowpass filtered and the mean value was calculated between 1 and 25 s after the end of microstimulation. We called this the mean rectified BS EMG (BS rEMG). For BS EMG burst frequency calculation, the time interval between the start of the 1^{St} burst and the end of the 5^{th} burst were determined visually. For the Δp_{(SV)} and Δp_{(VD)} maximal amplitude we determined the maximum value for Δp_{(SV)} and Δp_{(VD)} between 0.5 and 4 s after the end of microstimulation. Data fitting was done in Excel (Microsoft Inc., USA) using a generalized reduced gradient (GRG2) algorithm. For graphical display, we removed stimulation artefacts from the EMG data. Presented values are given as means±standard error of the mean (SEM). To test statistical significance we used Student's t-test with a P-value <0.05 considered significant.

### Results

Brief electrical microstimulation of LSt neurons evoked ejaculation, the expulsion of semen at the urethral meatus, in 17 out of 17 anesthetized adult rats. In 10 out of the 17 rats, motile spermatozoa were observed by optical bright field microscopy. In the other 7 animals, the ejaculate contained immotile or no spermatozoa.

To further characterize ejaculation elicited by LSt neuron microstimulation, we quantified three critical parameters of ejaculation: i) SV contraction was recorded via SV luminal pressure change (Δp_{(SV)}), ii) BS muscle activity was recorded with a BS muscle electromyogram (BS EMG) and iii) VD contraction was recorded via VD luminal pressure change (Δp_{(VD)})). After the application of 60-100 current pulses (200 Hz) in the LSt neuron area at L4, the SV luminal pressure immediately rose and fell, followed by prolonged rhythmic contractions of the BS muscle (FIGS. 1 B and C). Δp_{(SV)} followed a smooth curve, reaching a maximum value of 4.05±0.64 mmHg, 1.34±0.08 s after the onset of LSt neuron microstimulation and with a half width of 1.24±0.04 s (n=12). BS EMG activity in the form of bursts (FIG. 1B, inset middle panel) started 3.2±0.08 s after the onset of LSt neuron microstimulation. Furthermore, 95% of the BS EMG activity had occurred at 25±2 s (N=23). Occasional BS muscle contractions were observed even ˜50 s after the end of LSt neuron microstimulation (asterisk in FIG. 1C). The first 5 bursts of BS muscle activity occurred at a frequency of 2.4±0.2 Hz (n=23). Similar burst-like behavior in the BS EMG has been observed in copulating and anesthetized rats during ejaculation. In separate experiments we observed an increase in VD luminal pressure elicited by LSt neuron stimulation (FIG. 1D). Δp_{(VD)} reached a maximum value of 9.8±0.91 mmHg, 0.66±0.03 s after the onset of LSt neuron microstimulation (n=5). The present data shows that brief LSt neuron stimulation suffices to sequentially activate the peripheral physiological events leading to emission and expulsion.

### 2-Light Stimulation

### Materials and Methods

### Viral Vector

The three pseudotypes of AAV (2/2, 2/5, and 2/8) tested in this study were provided by the laboratory of gene therapy, INSERM U649, Nantes, France. The AAV recombinant genome contains the coding sequence for GFP (green fluorescent protein of Jellyfish Aequorea Victoria) under the control of the cytomegalovirus (CMV) promoter and the bovine growth hormone (BGH) polyadenylation signal, flanked by AAV2 terminal repeats. These nucleotide sequences were inserted in a plasmid expressing AAV2, AAV5 or AAV8 capsid gene to form AAV 2/2, 2/5, and 2/8 pseudotypes, respectively. Plasmids were transfected into HEK293 cells and purified solutions (phosphate buffered saline containing Mg and Ca ions) of AAV were obtained with the final following titrations, as determined by dot-blot assay: AAV2/2, 1.12.10¹¹ vector genomes (vg)/ml; AAV2/5, 3.3.10¹² vg/ml; AAV2/8, 9.10¹¹ vg/ml.

### Chronic Spinalization

Six male Wistar rats were included in the spinalized group. They were anaesthetized with isoflurane (1.5-2%) while their body temperature was maintained at 37° C. using a homeothermic blanket. The skin and muscles over the midthoracic vertebrae were incised and small retractors were used to separate the muscles overlying the spinous processes of the thoracic (T6-T8) vertebrae. The T8 spinal cord was exposed through a laminectomy of the T7-T8 vertebrae. The dura was incised, 0.2 ml of xylocalne 2% was dropped over the incision, and after 2 min, a complete transversal section, the completeness of which was verified with the aid of a surgical stereoscope, of the underlying T8 spinal cord was performed. A sterile gelform sponge was then placed between the cut ends of the spinal cord. Finally, the overlying muscles and skin were sutured. Post-operative care, including antibiotherapy, was provided to spinalized rats until the end of the experiment.

### Intra-Spinal Injection

Intra-spinal injection procedure was conducted in aseptic conditions. For each pseudotype virus, 4 rats (2 spinalized and 2 intact) were included. Under pentobarbital anaesthesia (40 mg/kg i.p.), the spine was exposed dorsally and fixed in a stereotaxic frame. Laminectomy between vertebrae lumbar (L1) and T13 exposed spinal segment L4. After dura removal, the spinal cord was incubated 20 min with 3 units/ml collagenase type VII from Clostridium histolyticum to improve spinal access. Finely pulled glass micropipettes (tip diameter ˜70 μm) were set in a micromanipulator apparatus. A 50 μM diameter Formvar coated nichrome wire was glued parallelly to the micropipette for electrical microstimulation. Bipolar electrodes were implanted into the proximal portion of the bulbospongiosus muscle (BS) for electromyogram measurement. The tip of the micropipette was placed on the spinal cord dorsal surface, adjacent to the dorsal spinal artery, and lowered vertically to 1500 μm depth for targeting lumbar spinothalamic cells (laminae X and VII medial). A first electrical stimulation (10 μA, 0.5 ms duration biphasic rectangular current pulses applied in trains of 60-100 pulses at 200 Hz) was applied and the contractile response of BS was monitored on an oscilloscope. The micropipette was lowered by increment of 100 μm (1-4 motions; maximal depth 1900 μm), with stimulation repeated at each increment, until a rhythmic an intense BS response was observed on the oscilloscope. Once such as BS response was obtained, 1 μl of the viral solution containing 1.10⁸ vg in isotonic saline was delivered over 10 minutes using a hydraulic microdriving system. At the end of the injection, the micropipette was let in place for 5 minutes and then slowly removed from the tissue. The area of laminectomy was filled with agar solution to protect the spinal medulla and overlying muscles and skin were sutured. Animals were housed individually for 3 weeks until histological procedure.

### Histological Procedure

Rats were anaesthetized with pentobarbital (60 mg/kg, i.p.) and transcardially perfused with phosphate buffered saline (PBS) and then paraformaldehyde 4% (PAF). Spinal cord (L2-S1 medulla segments) and brain were collected in PAF 4% and, 3 hours after, were put in sucrose 30% for 2-3 days at 4° C. Tissue samples were then frozen in isopentane (−40° C., 3 min) and stored at −80° C. until slicing. Serial coronal 30 μm-thick sections of brain and spinal cord were performed using a cryostat. One series of slices was mounted in Vectashield medium for fluorescence visualization and another series was processed for cresyl violet coloration for anatomical identification.

### Transfection Analysis

For analysis of GFP native fluorescence, sections were visualized under epifluorescence illumination using fluorescein isothyocyanate (FITC) filter on a Nikon microscope. Pictures (20× Plan Fluor objective; same parameters of acquisition except varying time between 0.33 and 3 s) of transfected area were taken with a CCD camera and further analysed with NIS-Element software (Nikon). Cresyl violet stained sections, adjacent to GFP-positive sections, were used for localisation of transfected cells. Cells expressing GFP were counted and automatically delimited for measurement of fluorescence mean intensity and area. The total number of GFP-positive cells, the median of the cell mean fluorescence intensities, and the sum of cell areas were calculated for each pseudotype virus and each rat group. Spreading of the injection (estimated as the area of maximal density of GFP-positive cell bodies) and extension of GFP fluorescence (estimated as the area where GFP-positive neuropils were found) were determined for each pseudotype virus and each rat group. Lateral and dorso-ventral injection spreading was assessed on 3 slices containing the estimated site of injection.

### Results

Results were collected in 8 animals as follows: 4 AAV2/2 (2 intact and 2 spinalized), 2 AAV2/5 (2 intact), and 2 AAV2/8 injected rats (1 intact, 1 spinalized). Microscopic examination of brains, from the medulla oblongata to the frontal cortex, did not reveal GFP-positive elements.

In 5 cases, the site of injection was found centro-medial, in the vicinity of LSt cells, whereas in 3 cases, the site was located more ventrally, closed to the lamina VIII. In the rostro-caudal direction, injection spreading was limited to L3-L4 spinal segments and GFP-positive neuropils were found extending over 3-4 spinal segments (L2-L6) on both sides of the injection site (Table 1). In the dorso-ventral and lateral directions, injection diffusion was often restricted to the injection side but some GFP-positive cell bodies were detected on the other side, more particularly in AAV2/8 delivered animals. GFP-positive neuropils were also found on the side opposite to injection and sometimes crossing projections were identified. Again this observation was more frequent in AAV2/8 injected rats. Few GFP-positive cell bodies were observed outside the injection spreading area, with no noticeable differences from a pseudotype to another. For AAV2/2, rostro-caudal injection diffusion appeared of lesser extent in spinalized than in intact rats, whereas it was rather the contrary for AAV2/8 (Table 1). In intact rats, rostro-caudal injection diffusion was very similar from a pseudotype to another (Table 1). A difference between pseudotypes virus appeared for the dorso-ventral/lateral injection spreading with the following ranking: AAV2/8>AAV2/2>AAV2/5 (Table 1). Finally, the extent of GFP-positive neuropils was found smaller in AAV2/5 injected rats as compared to AAV2/2 and AAV2/8 (Table 1). It could be noticed that the range of GFP-positive diffusion seems reduced in spinalized rats in comparison with intact ones.

Although no staining of neuronal marker was performed, morphology and size of the cell bodies expressing GFP let us suggest that neurons constitute the main contingent of cells transfected by AAV pseudotypes virus. Counting of GFP-positive cell bodies revealed substantial differences between AAV pseudotypes (Table 1). The total number of cells expressing GFP in AAV2/8 injected animals was 3.9 and 1.7 times that determined in AAV2/5 and AAV2/2, respectively. The number of GFP-positive cells seemed lower in spinalized than in intact rats (Table 1). Cell mean fluorescence intensity (given in arbitrary units) was found comparable from a pseudotype to another, with less than 30% difference between AAV2/2 and AAV2/8 (Table 1). In addition, spinalization did not appreciably alter this parameter. Determination of the total area of GFP-positive cell bodies revealed a transfection area for AAV2/8 pseudotype 4.7 and 2.2 times larger than for AAV2/5 and AAV2/2, respectively (Table 1). It was noticed that this parameter was slightly diminished in spinalized animals.

In conclusion the AAV2/8 pseudotype is the best adapted viral vector for transfection of potentially high proportion of LSt cells.

Table 1

| | Injection spreading^{a} | | | | | |
|---|---|---|---|---|---|---|
| | (μm) | | | | | |
| | | Dorso- | | | Cell mean | Cell Total |
| AAV | Rostro- | ventral/ | | Transfected | intensity | area |
| pseudotype | caudal | Lateral | (mm) | cells number | (AU) | (μm²) |
| 2/2 | | | | | | |
| Intact | 2115 | 770 | 12.4 | 192 | 14.8 | 1.3.10⁵ |
| | (1530-2700) | (755-786) | (11.4-13.4) | (140-243) | (10.2-19.3) | (7.0.10⁴-1.8.10⁵) |
| Spinalized | 930 | 776 | 7.6 | 115 | 15 | 6.0.10⁴ |
| | (840-1020) | (752-801) | (7.4-7.7) | (98-132) | (14.6-15.8) | (4.9.10⁴-7.1.10⁴) |
| 2/5 | | | | | | |
| Intact | 2295 | 563 | 6.9 | 84 | 13.7 | 6.2.10⁴ |
| | (1980-2610) | (560-566) | (6.1-7.6) | (76-91) | (13.1-14.3) | (6.0.10⁴-6.5.10⁴) |
| Spinalized | ND | ND | ND | ND | ND | ND |
| 2/8 | | | | | | |
| Intact | 2160 | 935 | 11.6 | 324 | 11.4 | 2.8.10⁵ |
| Spinalized | 3240 | 877 | 9.8 | 199 | 13.6 | 1.6.10⁵ |

^{a}zone where the maximal density of GFP-positive cell bodies was found
^{b}zone where GFP-positive neuropils were found
Values are means or median (for cell mean intensity) with individual figures between brackets except for pseudotype 2/8. ND: not determined.

LSt cells are being transformed with photo-activable depolarizing channel (ChannelRhodopsin-2; ChR2) delivered to animals via AAV2/8 pseudotype.

LSt cells will then be activated through monochromatic light beam driven to LSt cells spinal area by implantable optic fibre, for triggering ejaculation.

### Materials and Methods

### Viral Vector

AAV2/8 pseudotype will be recombined to contain the following sequences: mammalianized synthetic form of ChR2 gene (Genbank accession N° EF474017) fused with GFP coding sequence (Genbank accession N° M62654) under the control of the neuron specific enolase promoter (150 base pairs 5′ to the start codon of the rat neuron specific enolase gene first exon; Genbank accession N° 019973) and the bovine growth hormone (BGH) polyadenylation signal. A CMV enhancer region will be added 5′ to promoter. The above sequences will be flanked by AAV2 inverted terminal repeats and co-transfected with helper plasmid into HEK293 cells. The helper plasmid will contain AAV2 rep and AAV8 cap genes with the required adenovirus helper genes including E4, VA, E2a helper regions. Recombinant AAV2/8 pseudotypes will be purified by iodixanol step gradients and Sepharose Q column chromatography and finally titrated by dot-blot assay.

### Surgical Procedures

At least 20 rats (10 intact and 10 spinalized) will be included in this set of experiments. Chronic spinalization and intra-spinal injection of the viral vector will be performed as described previously.

Three weeks after intra-spinal AAV2/8 delivery (10⁹ vg in ˜1 μl), rats will be subject to optical stimulation and recording of physiological markers of ejaculation. Rats will be anaesthetised with pentobarbital (40 mg/kg, i.p.) and their temperature maintained at 37° C. using a homeothermic blanket. The carotid artery will be catheterised to record blood pressure. The dorsal part of the right BS muscle will be implanted with bipolar steel wire electrodes for BS-EMG recording (physiological marker of the expulsion phase of ejaculation) and a catheter inserted into the prostatic portion of the vas deferens lumen for vas deferens intraluminal pressure measurement (physiological marker of the emission phase of ejaculation). BS-EMG and vas deferens pressure will be digitized and stored on a computer for further analysis. For photonic stimulation, a multimode optical fibre set in a micromanipulator apparatus will be slowly lowered in the spinal area where AAV2/8 was injected.

### Optical Stimulation

Stimulation of ChR2-expressing neurons will be accomplished using a multimode optical fibre coupled to a 473 nm diode pumped laser (20 mW output power). In order to find the optimal stimulation parameters, various light stimulation protocols will be applied (at least 200s interval between each stimulation) with varying time and intensity illumination while ejaculatory responses (BS-EMG and vas deferens pressure) are monitored. At the end of the experiment, rats will be taken for histological procedure and transfection analysis as already described.

1.-14. (canceled)

15. A method for eliciting ejaculation in a male individual, comprising delivering one or more stimulation pulses to lumbar spinothalamic (LSt) cells via a light stimulus, in an effective amount to activate LSt cells for achieving expulsion of sperm, wherein the LSt cells express a light-activated cation channel protein.

16. The method of claim 15, wherein the light-activated cation channel protein is ChR2, Chop2, ChR2-310, Chop2-310, or fragments or derivatives thereof.

17. The method of claim 15, wherein the light stimulus is provided by a xenon lamp, LED or a laser.

18. The method of claim 15, wherein the level of light intensity is from 0.1 mW/mm² to 500 mW/mm².

19. The method of claim 15, wherein the wavelength of the light stimulus is from 400 nm to 600 nm, or is suitable to activate the light-activated cation channel protein.

20. The method of claim 15, wherein the light stimulus is provided in a series of light pulses having a period from 0.1 ms to 1 ms.

21. The method of claim 15, wherein the light stimulus is provided by a wearable optical device.

22. The method of claim 15, wherein the light stimulus is provided by a wearable optical device being a light-emitting diode.

23. The method of claim 15, wherein the individual suffers from an ejaculation failure.

24. An adeno-associated virus of serotype 2/8 (AAV2/8) comprising a light-activated cation channel protein.

25. The adeno-associated virus of serotype 2/8 (AAV2/8) of claim 24, wherein the light-activated cation channel protein is ChR2, Chop2, Chr2-310 or Chop2-310.

26. The adeno-associated virus of serotype 2/8 (AAV2/8) of claim 24, wherein the light-activated cation channel protein is encoded by SEQ ID NO: 1 or SEQ ID NO: 2.


SEQUENCE LISTING (2 sequences)

SEQ ID NO: 1 - DNA, 930 bp, Chlamydomonas reinhardtii
atggattatggaggcgccctgagtgccgttgggcgcgagctgctatttgtaacgaacccagtagtcgtcaatggctctgtacttgtgcctgaggaccagtgttactgcgcgggctggattgagtcgcgtggcacaaacggtgcccaaacggcgtcgaacgtgctgcaatggcttgctgctggcttctccatcctactgcttatgttttacgcctaccaaacatggaagtcaacctgcggctgggaggagatctatgtgtgcgctatcgagatggtcaaggtgattctcgagttcttcttcgagtttaagaacccgtccatgctgtatctagccacaggccaccgcgtccagtggttgcgttacgccgagtggcttctcacctgcccggtcattctcattcacctgtcaaacctgacgggcttgtccaacgactacagcaggcgcaccatgggtctgcttgtgtctgatattggcacaattgtgtggggcgccacttccgccatggccaccggatacgtcaaggtcatcttcttctgcctgggtctgtgttatggtgctaacacgttctttcacgctgccaaggcctacatcgagggttaccacaccgtgccgaagggccggtgtcgccaggtggtgactggcatggcttggctcttcttcgtatcatggggtatgttccccatcctgttcatcctcggccccgagggcttcggcgtcctgagcgtgtacggctccaccgtcggccacaccatcattgacctgatgtcgaagaactgctggggtctgctcggccactacctgcgcgtgctgatccacgagcatatcctcatccacggcgacattcgcaagaccaccaaattgaacattggtggcactgagattgaggtcgagacgctggtggaggacgaggccgaggctggcgcggtcaac

SEQ ID NO: 2 - DNA, 930 bp, artificial
  EF474017 derived from Chlamydomonas reinhardtii
atggactatggcggcgctttgtctgccgtcggacgcgaacttttgttcgttactaatcctgtggtggtgaacgggtccgtcctggtccctgaggatcaatgttactgtgccggatggattgaatctcgcggcacgaacggcgctcagaccgcgtcaaatgtcctgcagtggcttgcagcaggattcagcattttgctgctgatgttctatgcctaccaaacctggaaatctacatgcggctgggaggagatctatgtgtgcgccattgaaatggttaaggtgattctcgagttcttttttgagtttaagaatccctctatgctctaccttgccacaggacaccgggtgcagtggctgcgctatgcagagtggctgctcacttgtcctgtcatccttatccacctgagcaacctcaccggcctgagcaacgactacagcaggagaaccatgggactccttgtctcagacatcgggactatcgtgtggggggctaccagcgccatggcaaccggctatgttaaagtcatcttcttttgtcttggattgtgctatggcgcgaacacattttttcacgccgccaaagcatatatcgagggttatcatactgtgccaaagggtcggtgccgccaggtcgtgaccggcatggcatggctgtttttcgtgagctggggtatgttcccaattctcttcattttggggcccgaaggttttggcgtcctgagcgtctatggctccaccgtaggtcacacgattattgatctgatgagtaaaaattgttgggggttgttgggacactacctgcgcgtcctgatccacgagcacatattgattcacggagatatccgcaaaaccaccaaactgaacatcggcggaacggagatcgaggtcgagactctcgtcgaagacgaagccgaggccggagccgtgcca